(19) United States
(12) Patent Application Publication
(10) Pub. No.: US 2006/0200151 A1
(43) Pub. Date: Sep. 7, 2006

(54) Orthopedic screw for use in repairing small bones

(76) Inventors: Dustin Ducharme, Akron, OH (US); Lee A. Strnad, Broadview Hts, OH (US); David B. Kay, Akron, OH (US)

Correspondence Address: HUDAK, SHUNK & FARINE, CO., L.P.A., 2020 FRONT STREET, SUITE 307, CUYAHOGA FALLS, OH, 44221, US

(21) Appl. No.: 11/369,631

(22) Filed: Mar. 7, 2006

Related U.S. Application Data
(63) Continuation-in-part of application No. 11/340,365, filed on Jan. 26, 2006.
(60) Provisional application No. 60/648,209, filed on Jan. 28, 2005.

(51) Int. Cl. A61B 17/58

(52) U.S. Cl. 606/73

(57) ABSTRACT

An orthopedic screw having a torque driving head with a rounded side a spherical wall for multiaxial use, a self starting, self tapping insertion tip and a threaded portion including a cancellous thread. The threaded portion has a major diameter defined by a spiraling thread and a minor diameter. The head is joined to the threaded portion by an area of from about 2 to about 6 turns of the thread along the longitudinal axis in which the minor diameter tapers by an angle of from about 4 to about 12° and the major diameter of the screw remaining substantially the same meaning that the major diameter is constant to about +/−0.05 mm along the length of the threaded portion. The screw includes a multilobe torque driving recess joined to a cylindrical recess. In a further embodiment, the screw has an insertion tip which is forms a portion of a sphere.

### CROSS REFERENCE

This application claims priority as a Continuation-in-Part of U.S. Ser. No. 11/340,365, filed Jan. 26, 2006 and Provisional Application Ser. No. 60/648,209, filed on Jan. 28, 2005

### FIELD OF THE INVENTION

The present invention relates to an orthopedic screw for use alone and with a plate to repair or reconstruct one or more small bones.

### BACKGROUND OF THE INVENTION

The field of orthopedic medicine has grown tremendously in the past fifty years as surgical techniques, implants and instrumentation have developed and been improved. The medical companies have tended to focus their attention on the largest market areas so that some areas of the body, such as the spine, knees and hips, have received intense focus from the large medical companies. While the small bones are frequently subject to the need for re-constructive surgery for example, as a result of trauma, to counteract the effects of aging or to repair congenital deformities, this area has typically not received the same degree of attention from the medical companies as joint replacement, trauma and spinal areas. Consequently, the products available to the small bone surgeon often represent scaled down versions of products designed for the large long bone market which are not adequate for the fine bones and delicate procedures required of the small bone surgeon. Additionally, while there is a wide variety in the exact shape and mass of all bones, these variations become more problematic in providing orthopedic implants for small bone applications since there is less room on and about the bone for the surgeon to place and fix the construct. These bones are finer and have less surface area for placement of an implant, and less mass for the placement of screws and as a result, individual variations become more problematic for implants of stock design.

One problem that needs to be avoided in the delicate environment of the small bone area is the interference of screws, with other screws, and with the function of ligaments and tendons. It may be desirable to design an orthopedic plate so that securing screws converge in order to cause compression or increase the pullout strength. It is difficult when a screw impinges on or conflicts with the desired placement of another screw. Some surgeons prefer bicortical fixation in which a screw is sized so that the distal end is secured in cortical bone giving the screw better purchase, however, other surgeons may prefer to avoid placing a screw so that it projects beyond the outer surface of the anchoring bone. These factors are complicated by the relative lack of soft tissue and the presence of ligaments and tendons in the small bone areas. Consequently, the less forgiving biological environment in which the small bone surgeon works requires greater procedural precision and calls for specialized implants and tools.

The present invention is designed to meet the specific needs of the small bone surgeon to facilitate effective and repeatable procedures which provide for ease of use and a range of function for this specific area of specialization. The present invention could serve for the treatment of a broad range of indications including relatively straightforward fracture repair following trauma in an otherwise healthy individual where screws are used alone or with plates to maintain the integrity of the bones while they heal, as well as for more complex surgeries such as reconstruction to correct congenital or age related deformation. Reconstruction often includes arthrodesis or partial or total fusion which involves removal of a joint and the use of a mechanical-biological construct to keep the bones immobile while fusion occurs. Further small bone surgeons may be called upon to achieve soft-tissue balancing by readjusting the length of tendons and ligaments or to reshape the bone itself through removal or repositioning in a procedure known as an “ostetomy”. In an aging or diabetic population, these procedures may also involve dealing with the difficulties of poor quality bone and/or compromised soft tissue.

These surgeons typically include sub-specialists such as hand surgeons and feet and ankle and podiatric surgeons, but can also include general orthopedic surgeons who may be called upon to perform procedures on the small bones.

The present invention provides a screw for use alone or as part of a construct which could include a plate. The screw is designed specifically for the small bone market, i.e. for use in bones distal to the elbow and knee, including, for example, the ulna, radius, tibia, fibula, as well as the metacarpals, metatarsals, talus, calcaneus and phalanges. The screw can be used in applications previously mentioned, for example those that require fixation within a single bone such as the stabilization of a fracture or the screw can be used across two or more bones so as to facilitate total or partial fusion.

The screws are self-starting, self-tapping screws including the option of partial or full cannulation. The internal recess provided by the partial or total cannulation can be used as a place to press fit a screw holder in an instrument or can be used for additional fixation, for example using a wire. The screws include a cutting end having at least one and preferably multiple flutes, and most preferably 2 or 3 flutes about a conical recess. The screws further include a cancelleous thread. The screw further has a partial taper of the minor diameter of about 5° to about 15°, and more preferably about 6° to about 10°, and most preferably about 8° over about the first 2 to about 6, and more preferably about 4 complete turns of the threads.

The screws further include a torque driving recess that may be a hexagon, a sinusoidal shape, or a modification of a sinusoidal (multilobed) shape. The recess can be of a constant size in the direction of the longitudinal axis, or can taper inward along the longitudinal axis of the screw toward the bottom of the recess. In addition, the head of the screw can include a rounded portion or spherical shaped head to permit multiaxial insertion, i.e. in a corresponding rounded or spherical recess in a countersunk screw hole in a plate or other construct. The screws can be provided in typical lengths for small bone use, i.e. from about 5 mm to about 25 mm and typically in lengths of 8, 12, 16, and 20 mm with a major diameter of about 3.5 mm. The screws can include a constant thread pitch as shown, in particular for use with a bone plate. A further embodiment of the screw for use in fixation by itself is a screw which includes a compression thread which increases in the number of turns over a given length. This variable pitch will preferably be used for the thread over about half of the distal end of the screw. The screws can be made of appropriate biocompatible material, including for example surgical grade stainless steel and titanium.

In a still further embodiment, the screws can include the previously described thread, and or can have a smooth shaft (in which case, they may be referred to as “pegs”, although “pegs” which are often used to support bone and sometimes also include bone threads, as well as locking threads). In addition, the screws and pegs of the present invention can have a threaded head for locking with the threads of a construct or they can have a functional head which might include camming flanges or wings, for example for use in a variable axis locking mechanism assembly, or they might include a smooth rounded head for variable angled placement with regard to the screw hole in a construct, such as a plate. In this embodiment, the screws or pegs include a blunt insertion tip, which is preferably rounded and more preferably forms a substantial part of a sphere, such as at least about ⅓, and more preferably at least about 40% or even about a full half of a sphere. In this embodiment the insertion tip preferably is free from sharp edges, such as cutting flutes.

### BRIEF DESCRIPTION OF THE DRAWINGS

FIG. 1 is a side view of an orthopedic screw in accordance with the invention;

FIG. 2 is a cross-section of the screw taken along line 2-2 of FIG. 3;

FIG. 3 is a top perspective view of the screw of FIG. 1;

FIG. 4 is a bottom perspective view of the screw of FIG. 1;

FIG. 5 is a top view of a plate which could be used with the screw of the present invention;

FIG. 6 is a cross section taken along line 6-6 of the plate shown in FIG. 5;

FIG. 7 is a top view of a second plate which can be used in an assembly with the screws of the present invention;

FIG. 8 is a side view of the plate of FIG. 7;

FIG. 9 is a top view of a third plate which can be used with the screws of the present invention;

FIG. 10 is a side view of the plate of FIG. 9;

FIG. 11 is a detail of the head of the plate of FIG. 9 with the locking cam inserts in position in the holes which provide for a variable axis locking mechanism assembly;

FIG. 12 is a top view of a fourth embodiment of a plate which can be used with the present invention;

FIG. 13 is a side view of a second embodiment of an orthopedic peg in accordance with the invention;

FIG. 14 is a cross-section of the- peg of FIG. 13 taken along line 14-14 of FIG. 15;

FIG. 14a is a detail of the locking threads of the peg of FIG. 14;

FIG. 15 is a top view of the peg of FIG. 13;

FIG. 15a is a detail of the torque receiving recess shown in FIG. 15;

FIG. 16 is an end view of the insertion tip of the peg of FIG. 13;

FIG. 17 is a side view of a third embodiment of an orthopedic peg in accordance with the invention;

FIG. 18 is a cross-section of the peg of FIG. 17 taken along line 18-18 of FIG. 20;

FIG. 19 is a cross-section of the peg of FIG. 18 rotated by 90°;

FIG. 20 is a top view of the peg of FIG. 17;

FIG. 21 is an end view of the insertion tip of the peg of FIG. 17;

FIG. 22 is a top view of the locking cam insert which can be used in the variable axis locking mechanism assembly along with the pegs of FIGS. 17-21 and 24-27

FIG. 23 is a cross-section of the locking cam insert shown in FIG. 22;

FIG. 24 is a side view of a fourth embodiment of an orthopedic peg in accordance with the invention;

FIG. 25 is a cross-section of the peg of FIG. 24 taken along line 25-25 of FIG. 26;

FIG. 25a is a detail of the locking threads of the peg of FIG. 25;

FIG. 26 is a top view of the peg of FIG. 24;

FIG. 27 is an end view of the insertion tip of the peg of FIG. 24;

FIG. 28 is a side view of a fifth embodiment of an orthopedic peg in accordance with the invention;

FIG. 29 is a cross-section of the peg of FIG. 28 taken along line 28-28 of FIG. 30;

FIG. 29a is a detail of the locking threads of the peg of FIG. 28;

FIG. 29b is a detail of the screw threads of the peg of FIG. 28;

FIG. 30 is a top view of the peg of FIG. 28;

FIG. 30a is a detail of the torque receiving recess shown in FIG. 30;

FIG. 31 is an end view of the insertion tip of the peg FIG. 28;

FIG. 32 is a side view of a sixth embodiment of an orthopedic screw in accordance with the invention;

FIG. 33 is a cross-section of the screw of FIG. 32 taken along line 33-33 of FIG. 34;

FIG. 33a is a detail of the thread shown in FIG. 32;

FIG. 34 is a top view of the screw of FIG. 32;

FIG. 35 is an end view of the insertion tip of the screw of FIG. 32;

FIG. 36 is a side view of a seventh embodiment of an orthopedic screw in accordance with the invention;

FIG. 37 is a cross-section of the screw of FIG. 36 taken along line 37-37 of FIG. 38;

FIG. 37a is a detail of the locking threads of the screw of FIG. 36;

FIG. 37b is a detail of the screw threads of the screw of FIG. 37;

FIG. 38 is a top view of the screw of FIG. 36;

FIG. 38a is a detail of the torque receiving recess shown in FIG. 38; and

FIG. 39 is an end view of the insertion tip of the screw of FIG. 36;

### DETAILED DESCRIPTION OF THE INVENTION

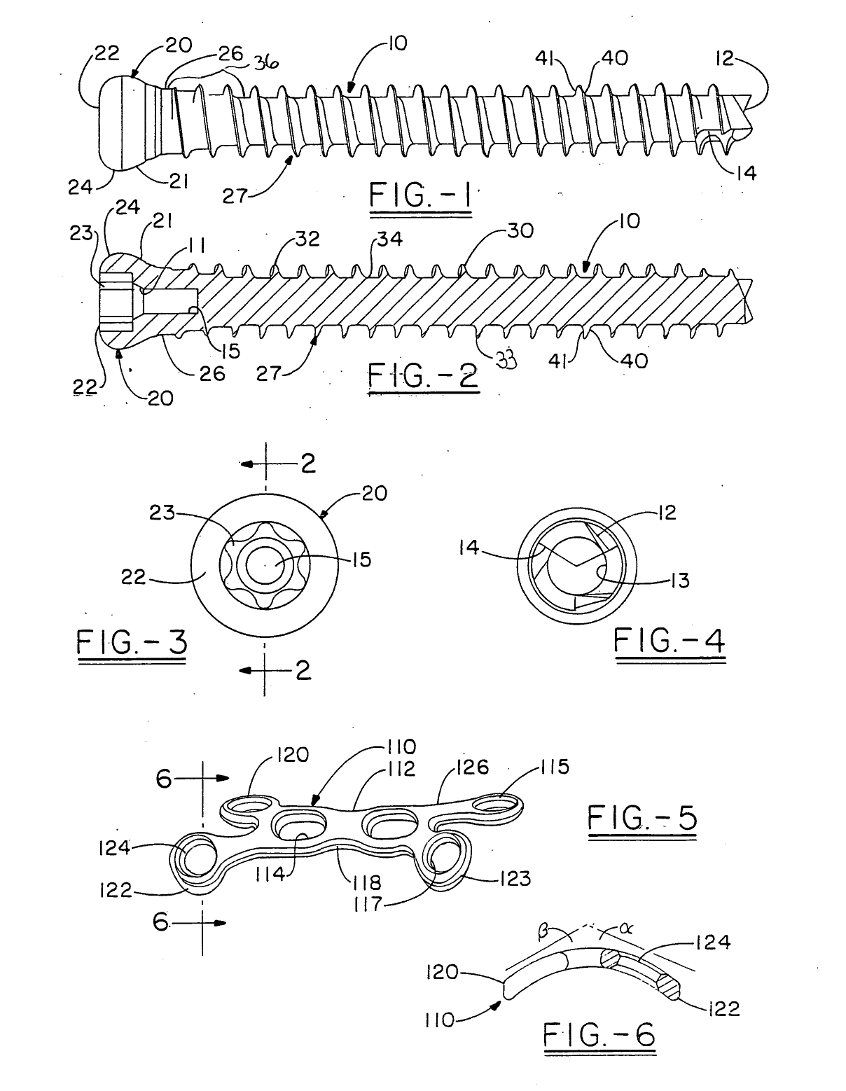

FIGS. 1-4 show an orthopedic screw 10 in accordance with the present invention. The distal end of the screw includes a cutting tip 12 which is self-starting and self-tapping. The term “distal” is used herein to mean the end that would be farthest from the point of attachment to a plate if one were used, i.e. the insertion tip, and “proximal” is used to mean the opposite end of the screw, i.e. the head. The cutting tip 12 is provided by a conical recess 13 and a plurality of flutes 14 or grooves that form sharp cutting surfaces at the terminus of the screw. The screw 10 can include a partial or full cannula 15 along its longitudinal axis. While the screw is shown as including a cannula in the form of a through bore in the drawings, the bore can project only partially toward the distal end of the screw, or can be absent. In a preferred embodiment the screw includes the partial cannulation which is a cylindrical recess extending at least about 1.5 mm up to about 5 mm, and preferably about 2 mm to 4 mm based upon the diameter of the screw. An angled area 11 connects the cannulation or recess with the torque driving recess. The canulation is used with a torque driving instrument that has a corresponding shaped post that will fit in the screw so that the screw is self-centering, is held in position on the torque driving instrument in a friction fit, and seats the screw so as to avoid stripping the interface between the screw and the torque driver.

The head 20 of the screw includes a rounded area 21 which preferably includes from about 0.75 mm to about 2.0 mm of a sphere having a diameter of from about 4 mm to about 5 mm. This defines a side wall which will allow for multi-axial placement in a screw hole, for example, in a plate that has a corresponding concavity. In the event that the screw is used alone, the rounded area eliminates sharp transitions between the threaded area and the head of the screw.

The screw head 10 has a relatively flat proximal surface 22 having radiused transitions 24 into the rounded area 21 of the side wall of the head. The proximal surface includes a torque driving recess 23, such as a modified multilobe shape as is shown in FIG. 3. A necked area 26 joins the rounded area 21 of the head side wall to a threaded portion 27 of the screw. The threaded portion 27 includes a cancellous thread 30 with a constant major diameter 32 which is defined by the spiraling outer edge of the thread 33 and a minor diameter 34 defined by the inner portion of the screw at the base of the thread. The minor diameter 34 is constant over a distal portion of the thread so as to define a cylinder with a spiraling thread. The minor diameter also includes a proximal portion that tapers inward over the length of the first four threads toward the distal end in order to improve fatigue strength and to improve compression at the proximal cortical bone interface and to compensate for bone re-adsorption. The tapered portion of the screw 36 includes a taper of from about 2° to about 20°, or more preferably from about 4° to about 12°, and most preferably about 6° to about 10° (i.e. about 8°) which tapers over from about 2 to about 10, and more preferably about 3 to about 6 complete turns (360°) of the thread 30. The pitch is between about 0.5 and 2.0 millimeters in length (i.e. a thread revolution of 360° per 0.5 to 2.0 millimeters).

The thread is a cancellous thread with a front thrust 40 surface having an angle of from about 10° to about 30°, or more preferably from about 15° to about 25°, and most preferably about 18° to about 22° (i.e. about 20°) to a plane perpendicular to the longitudinal axis of the screw, while the rear surface 41 forms an angle of about 0° to about 10°, or more preferably from about 0° to about 8°, and most preferably about 3° to about 7° (i.e., about 5°) to the plane perpendicular to the longitudinal axis of the screw.

The screw can be made from an appropriate biocompatible material having appropriate strength characteristics including surgical grade stainless steel or titanium or absorptive materials.

A plate with which the screw of the present invention can be used to advantage is shown in FIGS. 5 and 6. The plate 110 is shown having a modified x-shape or asymmetrical dog-bone shape with a central trunk portion 112 defining the longitudinal axis of the plate. The trunk portion 112 includes one or preferably more elongated screw holes 114 along the longitudinal axis. The number of screw holes will depend on the length of the plate, and may range from 0 to about 6. The screw holes 114 are preferably elongated to allow the plate to be set initially and subsequently to be slid into a different position and tightened down. Further, the screw holes include annular rings 115 of increased thickness in the vertical direction about through bores 117. The through bores 117 in the trunk portion have a longitudinal axis that is perpendicular to plane tangent to the top radius of the plate. The area linking the screw holes has a decreased width so as to define a waist area 118 that will bend laterally (or “curve”) relative to the longitudinal axis and which will bend longitudinally to form a curved area in and out of the plane of the plate. This thinner area also facilitates twisting of the plate so as to allow the plate to spiral, or wrap around it longitudinal axis. The increased annular area around the screw holes resists deformation when a bending device is used to apply a force to the plate through the screw holes.

The plate 110 also includes at least one set, and preferably two opposing sets of arms 120. As viewed in FIG. 5, these sets of arms can be viewed as a set of upper 122 and lower arms 123, although it is understood that the orientation of the plate can vary even after the plate has been fixed to the bone so that the terms upper and lower are only used to distinguish the pair on one side of the trunk portion 112 from the pair on the other side of the trunk portion 112. Each of the arms in a set includes screw holes 124 which are placed at a radially equal distance but which diverging asymmetrically from the longitudinal axis of the plate 110. More specifically, each set of arms includes one arm that defines a smaller angle of divergence a from the longitudinal axis of the trunk portion than the angle of divergence of the other arm β. For example, the first angle shown in FIG. 1 at α may be from about 5° to about to 25°, and more preferably from about 10° to about to 20° and most preferably from about 12° to about to 16°, while the second angle shown at β from about 10° to about to 35°, and more preferably from about 15° to about to 30° and most preferably from about 22° to about to 26° with a preferred difference in the angles beings from about 2° to about to 20°, and more preferably from about 4° to about to 16° and most preferably from about 8° to about to 12°. On the inferior side, or the side that would be facing the bone surface in use, the arms continue the radius of curvature of the trunk portion. The superior or top side of the plate has a similar radius of curvature as the top surface of the plate has an outline that corresponds with the shape of the bottom of the plate (excluding the thickened annular area surrounding the screw holes.) The screw holes 124 are placed with the longitudinal axis perpendicular a tangent to the top surface of the arm with the effect that the longitudinal axes of the screws converge in the direction of the distal end. This increases the pull-out strength of the screws. Since the arms are asymmetrical relative to each other, and in particular since they diverge from the longitudinal axis of the trunk portion at differing angles, conflicts in the positions of paired screws are avoided so that the screws of a set of arms do not impinge on each other. This is even more important in instances where the plate is bent around the longitudinal axis so as to wrap around the longitudinal axis of the bone.

The arms 120 also each include a screw hole 124 which, like the trunk portion 112 has a linking portion 126 that joins annular areas 125 of increased thickness that rings a through bore 127. Again this design facilitates the desired bending while resisting deformation of the screw holes 124 when they are used with the bending instrument to contour the plate. The angle of the arms 120 of each one of a pair of a respective set of arms 122 and 123 varies so as to create a bilateral asymmetry, meaning that the plate is not symmetrical with respect to a plane that passes through the longitudinal axis in the vertical direction from the superior (the top side relative to the bone) to the inferior side (the side facing the bone), the “first plane”. However, the position of the arms in each set is preferably flipped so that the symmetry about a plane transverse to the first plane is a mirror image, defined herein as transverse mirror symmetry. Further the length of each of the arms of a pair will vary so that the radial length of the center of the screw hole to the intersection with the longitudinal axis will be the same. As shown in FIG. 6, the plate includes a radial curve about the longitudinal axis. The radius is typically about 10 mm with a transverse dimension from the edge of one arm to the edge of the other arm of an upper or lower pair being about 15 or 16 mm, and the screw bore having a longitudinal axis of about 24° to an plane passing through the longitudinal axis of the plate. The bores are typically about 3.75 mm for a 3.5 mm diameter screw. In a further embodiment, the bore is threaded.

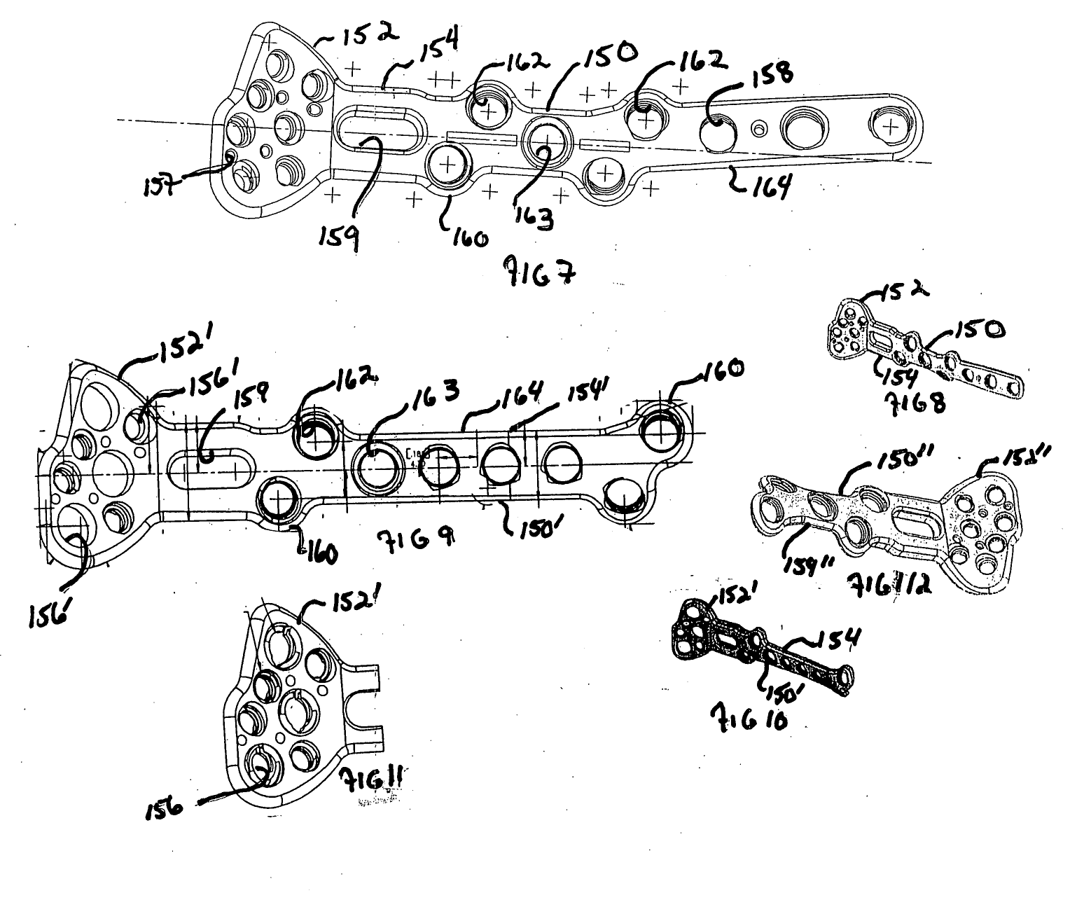

The screws and pegs of the present invention can be used with various other plates and implants and examples of various plate styles are illustrated in FIGS. 7 through 12. In particular, FIG. 7 shows a first embodiment of a small bone plate 150 that has a head portion 152 and a proximal plate portion 154. The head 152 includes bores 156 including internal threads, which receive the pegs of the invention. The proximal portion of the plate includes screw holes 158 which are also threaded and which receive the screws of the invention. The head may include additional holes 157 for guide wires for fixation or for use in placement or distraction during the surgical procedure. The plate can further include a slot 159 for viewing the surgical site, or for applying a compression. In the embodiment of FIG. 7, the proximal portion of the plate has two tabbed areas 160 which include offset holes 162 which provides for convergent fixation. This portion of the plate also includes a central hole 163. The plate also includes a tail portion 164 which extends along the bone. FIG. 8 illustrates the embodiment of FIG. 7 from a top and side perspective, and in which the fully contoured topography can be seen including the angle of the head and the spiral of the proximal portion of the plate. FIG. 9 illustrates a slightly different version of this elongated plate embodiment 150′ in which the tail portion curves less away from the longitudinal axis and is bounded on either end by the tabbed areas having the offset screw holes 162. This version also has a different head 152′ in which three of the holes 156′ in the head are enlarged and not threaded and thus intended to form a part of a variable axis locking mechanism assembly. FIG. 12 shows yet another embodiment of this plate 150″ having a shortened version of the proximal plate portion 154″ and a head 152″ which may correspond to either the fixed angle head shown in FIG. 7 or may include the variable angle axis holes of FIG. 11.

FIGS. 13 through 16 show a smooth locking peg which can be used for example in the head of the distal radius plate shown in FIGS. 7 through 12. In particular, these pegs 200 include a smooth shaft portion 201, and a head 202 having an external locking thread 203. The head further includes a torque receiving recess 205 which is similar to that described for the first embodiments of the screws in accordance with the invention, and further including means to retain the peg on a torque driver. Specifically this includes a bore 206 that receives a post on the driver.

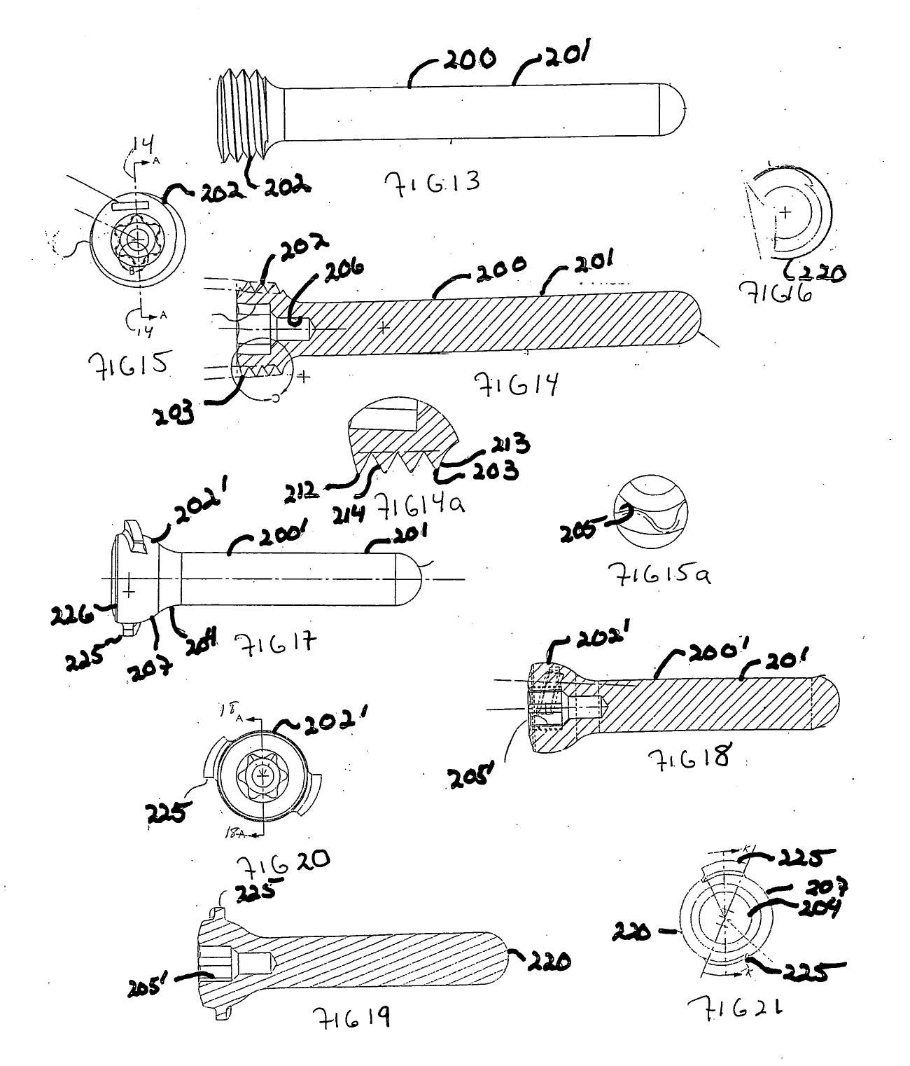

The locking threads 203 are best viewed in the detail shown in FIG. 14a, and include a radial edge 212, a front thrust face 213, and a trailing face 214. The angle of the front thrust face 213 is the same as the angle of the trailing face relative to a plane which transverses the longitudinal axis of the screw, and is about 30°+/−5° for each angle. Thus, the locking thread 203 on the head 202 of the peg is a symmetrical v-shaped thread when viewed in profile in cross section. The head 202 tapers along the longitudinal axis, in both the major and the minor diameter, by a similar amount, as is shown in FIG. 14. For the peg shown, the taper is 7°, or 3.5° per side when measured in cross-section.

Further, as is common to this and the other new embodiments shown, the peg has a blunt insertion tip 220 which preferably is rounded, and more preferably forms some portion of a sphere, i.e. at least about 25%, more preferably at least about 30% and most preferably 40 or 50% of a sphere having the same diameter as the cylindrical shaft 201 so that there is a smooth and seamless transition between the shaft and the insertion tip. Thus, if some portion of the peg protrudes beyond the far cortical surface, the tip does not provide for irritation of the surrounding flesh.

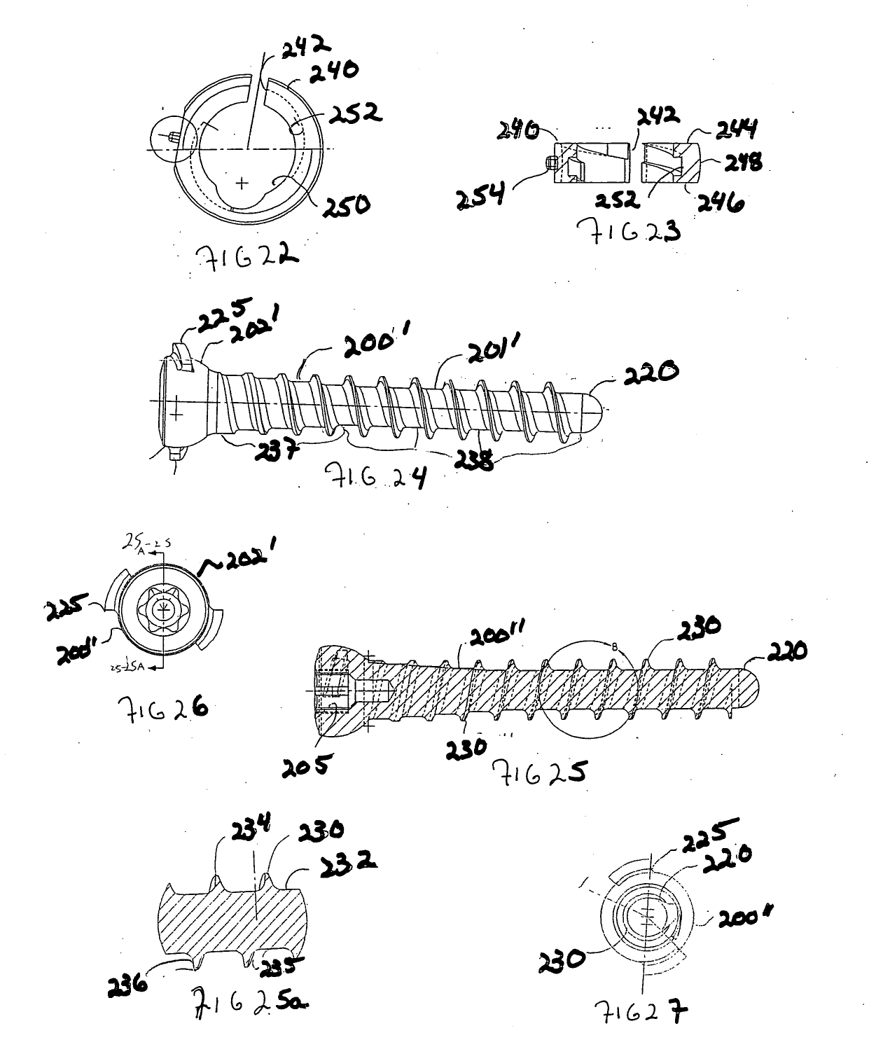

A variation on the smooth shaft peg of FIG. 13 is illustrated in FIGS. 17 through 21. This peg 200′ also includes the shaft 201 joined by a necked area 204 to the lower rounded area 207 of head 202′ which has camming flanges 225. The head has a relatively flat proximal area 226 which includes the torque receiving recess 205 as previously described. A third variation of this peg 200″ is shown in FIGS. 24 through 27. In this case, the peg includes a similar variable angle axis locking mechanism head 202′ as is shown in FIGS. 13 through 17. The head includes the camming flanges 225 previously described, and a torque receiving recess 205, also previously described. The peg also includes a shaft 201′ which includes a screw thread 230 which is better illustrated in FIG. 25a. The shaft 201′ has a minor diameter 232 about which the thread 230 spirals. The thread 230 includes a spiraling radial edge 234 best viewed in the thread detail FIG. 25a, which defines the major diameter. The thread further includes a front thrust face 235 which forms an angle of about 20°+/−5° to a plane transverse to the longitudinal axis of the screw. The trailing face 236 of the thread 230 forms an angle of about 5°+/−2° to the same plane. The shaft shown in FIGS. 24 through 27 has a taper in the minor diameter 232 over a portion 237 of the shaft 201′, such as the first three turns of the thread. Thereafter, the terminal portion 238 of the shaft 201′ has a constant minor diameter 232.

FIGS. 22 and 23 illustrate a locking cam insert 240 that can be used with the screw or pegs of the present invention. The camming insert 240 is a generally circular or ring shaped insert having an expansion gap 242 which is essentially a planar slice taken in the insert so as to create a gap, and thus a discontinuous ring. The insert 240 has a top surface 244 which is generally planar joined to a co-planar bottom surface 246 by an outwardly curving side surface 248. There is a concentric inwardly curving surface 250 which further includes the cam race 252, which in this case are two grooves that spiral a portion of the way down and around the inside surface. The grooves are open, and preferably only for a portion of the top 244 where the grooves are located. This open area of the race allows the cams to be introduced into the race. Subsequently, as the peg is turned in the camming insert, the cam engages the cam race and causes the insert to expand at the gap. This action causes the insert to lock in the recess in the plate which receives the insert. Further, the insert 240 includes a stop 254. The stop is a projection that is received in a well in the recess which retains the stop 254 and prohibits the cam insert from turning with the peg as it is turned relative to the plate.

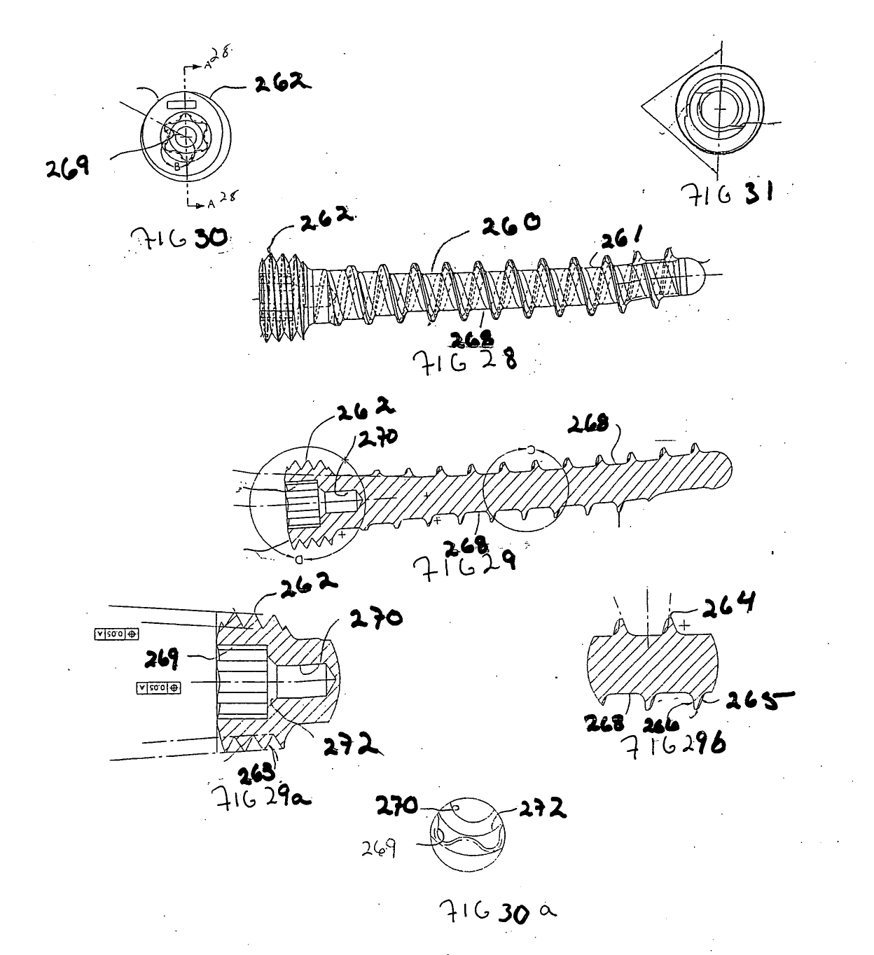

FIGS. 28 through 31 illustrate a version of the peg in accordance with the present invention which combines the locking head 202 shown in FIGS. 13 through 15 with the threaded shaft 201′ of FIGS. 24 through 27. Thus, the peg 260 shown in FIGS. 28 through 31 has a locking head 262 having a locking thread 263 as previously described except that the major and the minor diameter both have a taper of about 10° per side in cross-section for a total of about 20° of taper. Further, the shaft 261 has a thread 264 as described for the peg shown in FIG. 24a with a front thrust face 265, and a rear trailing face 266. The shaft further has a minor diameter 268 which has a taper over the first several, and preferably the first about 3 to about 5 turns of the thread. The head has a torque receiving recess 269 which has a modified sinusoidal curve. The recess includes a partial bore 270 that receives a post on the torque driver, and which is connected to the torque receiving recess 269 by a tapering transition 272.

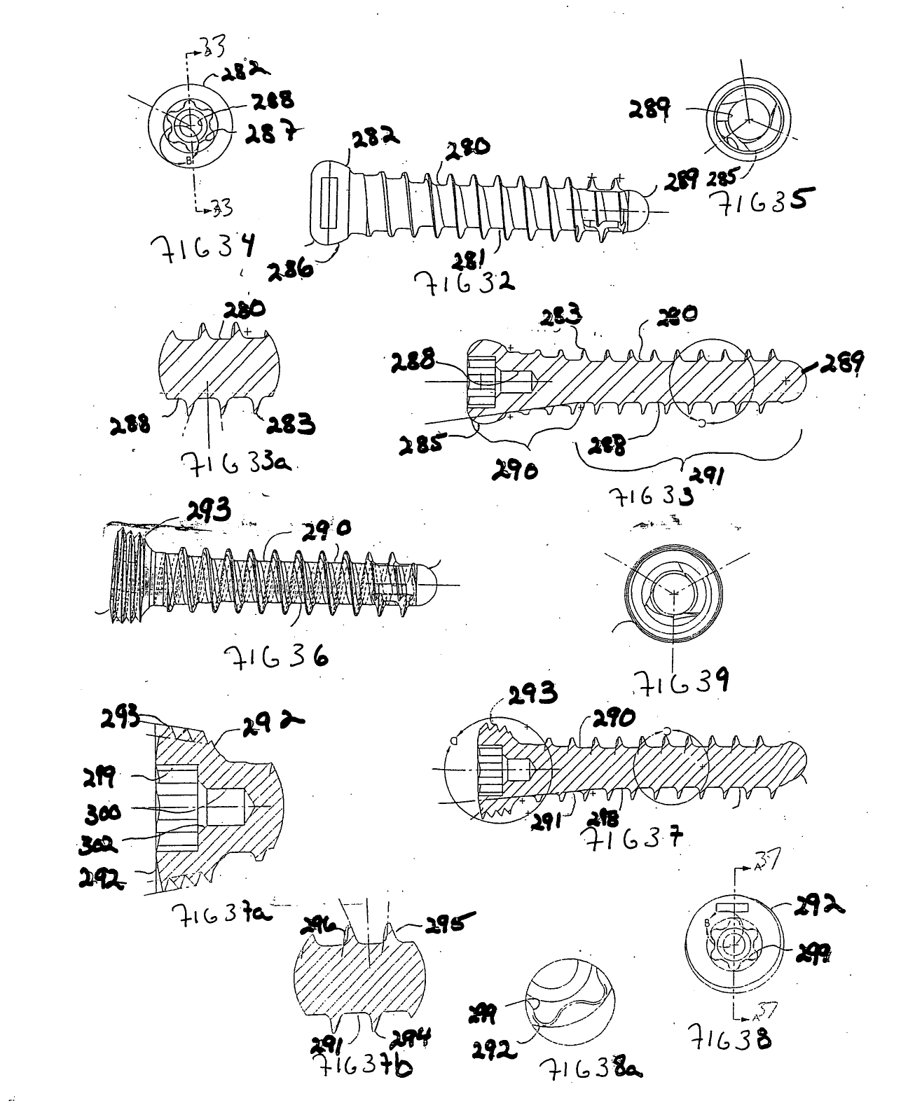

FIGS. 32 through 39 illustrate a version of the screw 280 of the present invention having a shaft 281 with a thread 283 and a minor diameter 288 that tapers over a portion 290 of the length of the shaft 281. The remainder of the shaft 291 has a constant minor diameter. The head 282 of the screw has a smooth side surface 285 with a rounded bottom area that allows for an angled placement in a screw bore. The head further includes a torque receiving recess 287 with a partial bore 288 that can be used to retain the screw on a screw driver. The screw has a blunt insertion tip 289.

FIGS. 36 through 39 illustrate a version of the screw in accordance with the present invention which combines the locking head 202 shown in FIGS. 13 through 15 with the threaded shaft 201′ of FIGS. 24 through 27. Thus, the screw 290 shown in FIGS. 36 through 39 has a locking head 292 having a locking thread 293 as previously described where that the major and the minor diameter both have a taper of about 3.5° per side in cross-section for a total of about 7° of taper. Further, the shaft 291 has a thread 294 as described for the peg shown in FIG. 24a with a front thrust face 295, and a rear trailing face 296. The shaft further has a minor diameter 298 which has a taper over the first several, and preferably the first 3 to 5 turns of the thread. The head has a torque receiving recess 299 which has a modified sinusoidal curve. The recess includes a partial bore 300 that receives a post on the torque driver, and which is connected to the torque receiving recess 299 by a tapering transition 302.

While in accordance with the patent statutes, the best mode and preferred embodiment have been set forth, the scope of the invention is not limited thereto, but rather by the scope of the attached claims.

What is claimed is:

1. An orthopedic screw comprising:
a head, an insertion tip, and a threaded portion with a longitudinal axis and having a major diameter defined by a spiraling thread and a minor diameter, the head including a torque driving recess and joined to the threaded portion by an area of from about 2 to about 6 turns of the thread along the longitudinal axis in which the minor diameter tapers by an angle of from about 4° to about 12° and the major diameter of the screw remaining substantially the same along the length of the threaded portion, the insertion tip being blunt, the screw being made from surgical stainless steel or titanium.

2. The orthopedic screw as set forth in claim 1, wherein the head further includes a threaded portion.

3. The orthopedic screw as set forth in claim 1, wherein the insertion tip comprises a portion of a sphere.

4. An orthopedic screw, comprising:
a head including a torque driving recess and a shaft portion terminating in a insertion tip which comprises some portion of a sphere.

5. The orthopedic screw as set forth in claim 4, wherein the head of the screw further includes a rounded side wall.

6. The orthopedic screw as set forth in claim 5, wherein the shaft defines a longitudinal axis and further includes a thread and the thread has a front thrust surface which forms an angle of from about 10° to about 30° to a plane perpendicular to a longitudinal axis of the shaft and a trailing surface which forms an angle of from 0° to about 10° to a plane perpendicular to the longitudinal axis of the shaft.

7. The orthopedic screw as set forth in claim 6, wherein the front thrust surface forms an angle of from about 15° to about 25° to the plane perpendicular to the longitudinal axis of the shaft and the trailing surface forms an angle of from 0° to about 8° to the plane perpendicular to the longitudinal axis of the shaft.

8. The orthopedic screw as set forth in claim 6, wherein the front thrust surface forms an angle of from about 18° to about 22° to the plane perpendicular to the longitudinal axis of the shaft and the trailing surface forms an angle of from about 3° to about 7° to the plane perpendicular to the longitudinal axis of the shaft.

9. The orthopedic screw as set forth in claim 6, wherein the thread is a cancellous thread and the minor diameter tapers over from about 2 to about 6 turns of the thread.

10. The orthopedic screw as set forth in claim 9, wherein the tapers is from about 3 to about 25°.

11. The orthopedic screw as set forth in claim 4, wherein the torque driving recess is a multilobe recess which is joined to a cylindrical recess.

12. The orthopedic screw as set forth in claim 4, wherein the shaft is substantially smooth.

13. The orthopedic screw as set forth in claim 4, wherein the head further includes one or more camming flanges.
